(19) United States
(12) Patent Application Publication
(10) Pub. No.: US 2006/0128788 A1
(43) Pub. Date: Jun. 15, 2006

(54) Purification of tegaserod maleate

(76) Inventors: Santiago Ini, Haifa (IL); Anita Liberman, Tel-Aviv (IL)

Correspondence Address: KENYON & KENYON LLP, ONE BROADWAY, NEW YORK, NY, 10004, US

(21) Appl. No.: 11/255,046

(22) Filed: Oct. 19, 2005

Related U.S. Application Data
(60) Provisional application No. 60/620,732, filed on Oct. 19, 2004.

(51) Int. Cl. A61K 31/405; C07D 209/14

(52) U.S. Cl. 514/419; 548/505

(57) ABSTRACT

Provided is a process for the purification of tegaserod maleate. Also provided is an isolated compound and the preparation thereof. This compound may be used as a reference marker and a reference standard, in the analysis of the purity of the tegaserod maleate.

### RELATED APPLICATIONS

The present application claims the benefit of U.S. Provisional Patent Application No. 60/620,732 filed on Oct. 19, 2004, which is incorporated herein by reference.

### FIELD OF THE INVENTION

The present invention relates to a process for the purification of tegaserod maleate.

### BACKGROUND OF THE INVENTION

Tegaserod maleate is an aminoguanidine indole 5HT4 agonist for the treatment of irritable bowel syndrome (EBS), and is marketed as Zelnorms.

The chemical name for Tegaserod maleate is: 1-(5-Methoxy-1H-indol-3-ylmethyleneamino)-3-pentylguanidine monomaleate.

The chemical structure of Tegaserod maleate is:

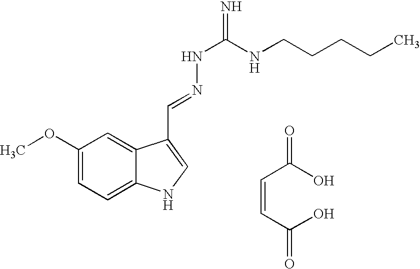

Tegaserod maleate is disclosed in the U.S. Pat. No. 5,510,353 and in its EP equivalent 505322 B1 (example 13 in both of them). The patent also describes the preparation of tegaserod base by reacting indole-3-carbaldehyde and aminoguanidine in a protic solvent in the presence of inorganic or organic acid (example 2a describes the reaction in methanol and hydrochloric acid). Although the melting point for tegaserod maleate is given (190° C. in Table 1, example 13), no information about its preparation and purification is provided.

The process described in U.S. Pat. No. '353 provides tegaserod free base containing an impurity of Tegaserod (TGS-dimer) at RRT 1.06, having the molecular weight of 6.17 g/mol. In table 1 (below) the analysis of the tegaserod base so obtained is described.

TABLE 1

| | | | | HPLC (%) | | |
|---|---|---|---|---|---|---|
| | | | | | | RRT = 1.06 |
| | | | | | | M.W = 403 |
| Entry | Solvent | Additive | Sample | MICHO | TGS | g/mol |
| 1 | MeOH (RT) | HCl | crude | 1.27 | 77.83 | 6.17 |

A process for preparing tegaserod maleate is described in co-pending U.S. application Ser. No. 11/115,871 filed on Apr. 26, 2005, which comprises reacting N-amino-N′-pentylguanidine hydroiodide (AGP-HI) with 5-Methoxy-1H-indole-3-carbaldehyde (5-MICHO) in water or an organic solvent in the presence of maleic acid to precipitate tegaserod maleate, with the proviso that another acid is not used.

### SUMMARY OF THE INVENTION

In one embodiment, the present invention relates to a method of purifying Tegaserod maleate comprising: combining tegaserod maleate and a mixture of a first organic solvent; adding an inorganic base; maintaining the reaction mixture at a -temperature of from about room temperature to about the boiling temperature of the mixture, for a sufficient time to obtain a precipitate; combining the precipitate with a second organic solvent and a maleic acid with or without water at a temperature of from about room temperature to about the boiling temperature of the mixture for a sufficient time to obtain a precipitate; and recovering the Tegaserod maleate.

In another embodiment, the present invention relates to a method of purifyng Tegaserod maleate comprising: combining tegaserod maleate, and a mixture of a first organic solvent; adding an inorganic base and an organic carboxylic acid; maintaining the mixture at a temperature of from about room temperature to about the boiling temperature of the mixture, for a sufficient time to obtain a precipitate; combining the precipitate with a second organic solvent and a maleic acid with or without water; maintaining the mixture at a temperature of from about room temperature to about the boiling temperature of the mixture for a sufficient time to obtain a precipitate; and recovering the tegaserod maleate. The tegaserod maleate produced by the process of the present invention contains less than about 0.02% area by HPLC of the dimrner impurity and of the impurities at RRT 2.01 and RRT 0.89.

In another embodiment, the present invention provides an isolated compound, having the formula I;

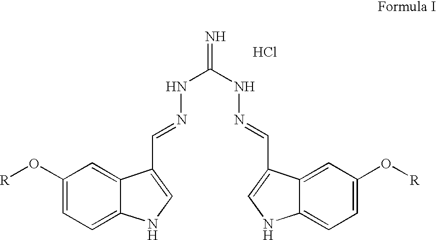

wherein R is selected from the group consisting of; saturated and unsaturated, branched and linear C₁-C₄ alkanes, C₁-C₄ ethers, C₁-C₃ alcohols, C₆-C₁₀ aromatic hydrocarbons and amides. Preferably, R is selected from the group consisting of: CH₂OCH₃, CH₂CH═C(CH₃)₂, CO—N(CH₃)₂, CH₃, C₂H₅, C₃H₇, benzoyloxy, CO-tert-C₄H₉, CO—N(C₂H₅)₂, CH₂—CO—N(CH₃)₂, CH₂—CH₂—N(CH₃)₂, (CH₂)₂—O—CH₃, (CH₂)₂—OH, isobutoxy, 2,3-di(OH)-propoxy and acetoxy. More preferably, R is methyl. -When R is a methyl group, the chemical name of this compound is: Bis-((5-Methoxyindol-3-yl)methylene) Carbonimidic dihydrazide Hydrochloride, and this compound is a tegaserod dimer (TGS-dimer).

TGS-dimer is characterized by ¹H NMR (500 MHz, DMSO d₆) δ(ppm): 11.73 (s, 2H), 8.53 (s, 2H), 7.89 (s, 4H), 7.75 (s, 2H), 7.37 (d, J=8.7 Hz, 2H), 6.87 (d, J=7.9 Hz, 2H), 3.87 (s, 6H); by ¹³C{¹H}NMR analysis (125 MHz) δ (ppm): 154.83, 152.03, 146.32, 132.18, 124.76, 112.73, 112.65, 110.48, 104.41, 55.56; by FAB mass spectrometry analysis showing the following data: m/z=404.1 ([M−H]⁺, 100%) and by IR(KBr) ν cm⁻¹: 3385 (N—H), 2944 (C—H), 1641, 1637, 1613 (C═N), 1528,1485 (Cₐᵣ-Cₐᵣ). The derivative may be used as an HPLC marker or as an indicator for purity in a process.

In yet another embodiment, the present invention provides a method for preparing the compound of formula I; 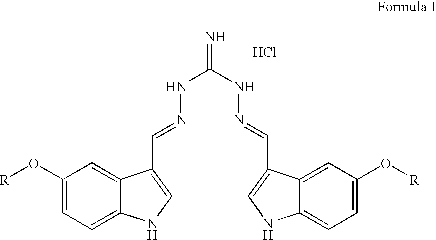
comprising the steps of;
a) mixing diaminoguanidine-HCl with water;
b) adding a compound of formula II; 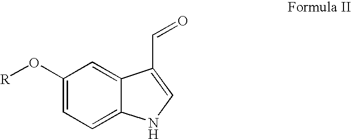
c) adding a mineral acid until a reaction mixture having a pH of about 2 to about 3 is obtained;
d) maintaining the reaction mixture at a temperature from about room temperature to about boiling temperature of the reaction mixture to obtain a precipitate; and
e) recovering a tegaserod derivative
wherein R is selected from the group consisting of; saturated and unsaturated, branched and linear C₁-C₄ alkanes, C₁-C₄ ethers, C₁-C₃ alcohols, C₆-C₁₀ aromatic hydrocarbons and amides. Preferably, R is selected from the group consisting of: CH₂OCH₃, CH₂CH═C(CH₃)₂, CO—N(CH₃)₂, CH₃, C₂H₅, C₃H₇, benzoyloxy, CO-tert-C₄H₉, CO—N(C₂H₅)₂, CH₂—CO—N(CH₃)₂, CH₂—CH₂—N(CH₃)₂, (CH₂)₂—O—CH₃, (CH₂)₂—OH, isobutoxy, 2,3-di(OH)-propoxy and acetoxy.
More preferably, R is methyl.
When R is a methyl group the chemical name of the compound in step b) is 5-methoxy-1H-indole-3-carbaldehyde (MICHO).

In yet another embodiment, the present invention provides a method for preparing crystalline forms of tegaserod maleate having an amount of the impurity TGS-dimer of less than about 0.02% by area percent HPLC comprising;
a) obtaining one or more samples of one or more tegaserod maleate batches;
b) measuring the level of tegaserod dimer in each of the samples of (a);
c) selecting the tegaserod maleate batch that comprises a level of the tegaserod dimer of less than about 0.02% by area percent HPLC based on the measurement or measurements conducted in (b); and
d) using the batch selected in (c) to prepare said tegaserod maleate.

Preferably, the tegaserod maleate is in a crystalline form.

### DETAILED DESCRIPTION OF THE INVENTION

As used herein the term “MICHO” refers to 5-Methoxy-1H-indole-3-carbaldehyde

As used herein the term “TGS” refers to Tegaserod.

As used herein the term TGS-dimer refers to Bis-((5-Methoxyindol-3-yl)methylene) Carbonimidic dihydrazide.

The present invention relates to a method of purifying tegaserod maleate Comprising: combining tegaserod maleate and a mixture of a first organic solvent; adding an inorganic base; maintaining the reaction mixture at at a temperature of from about room temperature to about the boiling temperature of the mixture, for a sufficient time to obtain a precipitate; combining the precipitate with a second organic solvent and a maleic acid with or without water at at a temperature of from about room temperature to about the boiling temperature of the mixture for a sufficient time to obtain a precipitate and recovering the Tegaserod maleate.

Preferably, the first organic solvent is a C₃ to C₇ ester. More preferably, the first organic solvent is isobutyl acetate or ethyl acetate.

Preferably, the mixture of the first organic solvent and water is 1:1 volumes.

Preferably, the mixture is maintained at room temperature, i.e., from a temperature of about 15° C. to a temperature of about 25° C.

Preferably, the inorganic base is an alkaline metal hydroxide or an alkaline earth metal hydroxide. More preferably, the inorganic base is selected from a group consisting of potassium hydroxide, sodium hydroxide, lithium hydroxide, magnesium hydroxide and calcium hydroxide. Most preferably, the inorganic base is sodium hydroxide.

Preferably, the inorganic base is added as an aqueous solution. Alternatively, it can be added as a solid, and than water is also added.

Preferably, the amount of inorganic base added is 10 mol equivalents.

Preferably the reaction mixture is maintained at said temperature for more than about 2 hours. More preferably, for about 24 hours.

Preferably, the second organic solvent is selected from the group consisting of: a C₁-C₈ alcohol, C₁-C₄ ketones, C₁-C₇ ethers, C₃-C₇ esters, acetonitrile and dioxane. More preferably, the second organic solvent is selected from the group comprising: methanol, ethanol, isopropanol, acetonitrile, butanol, acetone, dioxane, methyl ethyl ketone, tetrahydrofuran, ethyl lactate and ethyl acetate. Most preferably, the second organic solvent is ethyl acetate.

Preferably, combining the precipitate with a second organic solvent and a maleic acid is performed in the presence of water.

Preferably, the TGS maleate that is obtained by this method contains an amount of less than about 0.02% area by HPLC of the dimer impurity and of the impurities at RRT 2.01 and RRT 0.89.

One preferred process according to the present invention is depicted in the following scheme:

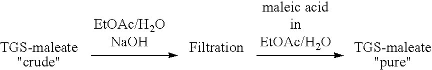

In another embodiment, the present invention relates to a method of purifying Tegaserod maleate comprising: combining tegaserod maleate, and a mixture of a first organic solvent; adding an inorganic base and an organic carboxylic acid; maintaining the mixture at a temperature of from about room temperature to about the boiling temperature of the mixture, for a sufficient time to obtain a precipitate; combining the precipitate with a second organic solvent and a maleic acid with or without water, at a temperature of from about room temperature to about the boiling temperature of the mixture for a sufficient time to obtain a precipitate and recovering the tegaserod maleate.

Preferably, the acid has pKa higher than about 2. More preferably the acid is C₂ to C₆ organic carboxylic acid. Most preferably, the acid is acetic acid.

Preferably, the first organic solvent is C₃ to C₇ ester, a C₁ to C₈ alcohol, a C₆ to C₁₂ aromatic hydrocarbon solvent, a C₁ to C₇ alkane or a C₂ to C₈ ether. More preferably, the first organic solvent is ethanol.

The inorganic base is preferably as described above.

Preferably, the mixture of the first organic solvent and water is 1:1 volumes.

Preferably, after adding the acid, the reaction mixture is maintained at said temperature for more than about 5 hours. More preferably, for about 24 hours.

Preferably, after adding the acid, the mixture is maintained at about room temperature Preferably, combining the precipitate with a second organic solvent and a maleic acid is performed in the presence of water.

Preferably, the precipitate is combined with the second organic solvent at a temperature of from about room temperature to about 85° C., preferably at about 70° C.

The recovering of the TGS maleate can be performed by any means known in the art, such as filtration.

The product obtained may be dried under suitable conditions. The product is preferably dried by heating at a temperature of about 30° C. to about 60° C., more preferably about 45° C. The drying is preferably carried under reduced pressure, more preferably a vacuum having a pressure of less than about 100 mmHg.

One preferred process according to the present invention is depicted in the following scheme:

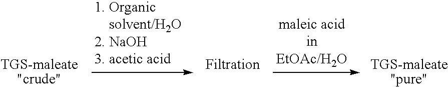

In particular, the method of the present invention is particularly suitable for reducing the TGS dimer impurity, and is also suitable for reducing the impurities having RRTs of 0.89 and 2.01. These impurities tend to discolour the final product The TGS maleate that is obtained by the present invention contains an amount of less than about 0.02% area by HPLC of the dimmer impurity and of the impurities at RRT 2.01 and RRT 0.89.

Table 2 (below) presents two examples in which the color in the starting material disappeared at the end of the process, and the level of TGS-dimer was reduced to less than 0.02% area by HPLC.

TABLE 2
Purification results of the TGS maleate

| | | | Impurity profile (%) | | | |
|---|---|---|---|---|---|---|
| Example | Yield | | RRT | TGS- | RRT | |
| No. | (%) | Color | 0.89 | dimer | 2.01 | Remarks |
| 1 | N/C | Yellowish | 0.04 | 0.06 | 0.05 | Start |
| | 91.20% | off white | <0.02 | <0.02 | 0.02 | End |
| 2 | N/C | Brownish | | 0.09 | 0.04 | Start |
| | 92.13% | Off white | | <0.02 | <0.02 | End |

The purified tegaserod maleate prepared according to the process described above, may be used for the preparation of tegaserod maleate crystalline forms.

The present invention provides an isolated compound having the formula I; 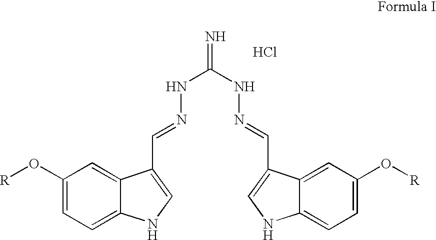
wherein R is selected from the group consisting of: saturated and unsaturated, branched and linear C₁-C₄ alkanes, C₁-C₄ ethers, C₁-C₃ alcohols, C₆-C₁₀ aromatic hydrocarbons and amides. Preferably, R is selected from the group consisting of: CH₂OCH₃, CH₂CH═C(CH₃)₂, CO—N(CH₃)₂, CH₃, C₂H₅, C₃H₇, benzoyloxy, CO-tert-C₄H₉, CO—N(C₂H₅)₂, CH₂—CO—N(CH₃)₂, CH₂—CH₂—N(CH₃)₂, (CH₂)₂—O—CH₃, (CH₂)₂—OH, isobutoxy, 2,3-di(OH)-propoxy and acetoxy. More preferably, R is methyl. When R is a methyl group, the chemical name of this compound is: Bis-((5-Methoxyindol-3-yl)methylene) Carbonimidic dihydrazide Hydrochloride, and this compound is a tegaserod dimer (TGS-dimer).

TGS-dimer is characterized by ¹H NMR (500 MHz, DMSO d₆) δ(ppm): 11.73 (s, 2H), 8.53 (s, 2H), 7.89 (s, 4H), 7.75 (s, 2H), 7.37 (d, J=8.7 Hz, 2H), 6.87 (d, J=7.9 Hz, 2H), 3.87 (s, 6H); by ¹³C{¹H}NMR analysis (125 MHz) δ (ppm): 154.83, 152.03, 146.32, 132.18, 124.76, 112.73, 112.65, 110.48, 104.41, 55.56; by FAB mass spectrometry analysis showing the following data: m/z=404.1 ([M−H]⁺, 100%) and by IR(KBr) ν cm⁻¹: 3385 (N—H), 2944 (C—H), 1641, 1637, 1613 (C═N), 1528, 1485 (Cₐᵣ-Cₐᵣ).

The present invention further provides a method for preparing the compound of formula I; 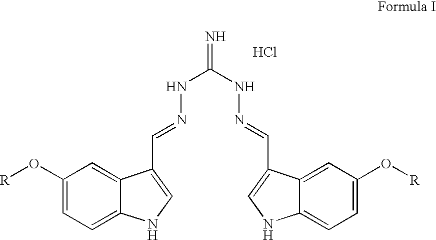
comprising the steps of,
a) mixing diaminoguanidine-HCl with water;
b) adding a compound of formula II; 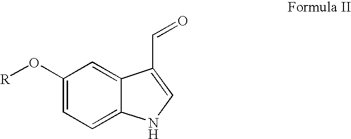
c) adding a mineral acid until a reaction mixture having a pH of about 2 to about 3 is obtained;
d) maintaining the reaction mixture at a temperature from about room temperature to about boiling temperature of reaction mixture to obtain a precipitate; and
e) recovering a tegaserod derivative,
wherein R is selected from the group consisting of: saturated and unsaturated, branched and linear C₁-C₄ alkanes, C₁-C₄ ethers, C₁-C₃ alcohols, C₆-C₁₀ aromatic hydrocarbons and amides. Preferably, R is selected from the group consisting of: CH₂OCH₃, CH₂CH═C(CH₃)₂, CO—N(CH₃)₂, CH₃, C₂H₅, C₃H₇, benzoyloxy, CO-tert-C₄H₉, CO—N(C₂H₅)₂, CH₂—CO—N(CH₃)₂, CH₂—CH₂—N(CH₃)₂, (CH₂)₂—O—CH₃, (CH₂)₂—OH, isobutoxy, 2,3-di(OH)-propoxy and acetoxy.
More preferably, R is methyl.

When R is a methyl group the chemical name of the compound in step b) is 5-methoxy-1H-indole-3-carbaldehyde [MICHO].

Preferably, the acid is selected form the group consisting of inorganic acids such as: HCl, HBr, H₃PO₄ and H₂SO₄ or an organic acid such as any carboxylic acid. Most preferably, the acid is HCl. Preferably, the reaction mixture is maintained at 70° C. for about 20 minutes to about 1 hour, more preferably for 30 minutes, and subsequently at about room temperature for about 10 hours to about 14 hours, more preferably overnight, while stirring.

The compound of formula II used in the above process is commercially available.

The following Scheme describes the formation of TGS-dimer

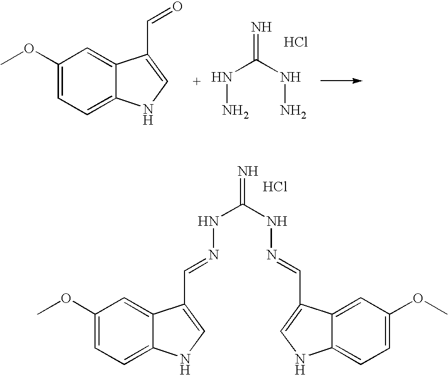

The isolated TGS-dimer of the invention can be used as a reference marker (purity marker) for TGS maleate. In the context of the present invention, a reference marker (purity marker) is a compound that is an impurity in a principal compound such as an active pharmaceutical ingredient (API). Detection or quantification of a reference marker by a suitable analytical technique establishes and defines the purity of, for example, the API; either in bulk, for example as obtained from synthesis, or as isolated from a pharmaceutical dosage form that includes the API. Manufacturing lot release criteria can be established with reference to a particular amount or concentration of a reference marker in the bulk product. Detection and quantification of the reference marker in the API of a pharmaceutical dosage form can serve as a measure of the shelf-life of the pharmaceutical dosage form.

Those skilled in the art of drug manufacturing research and development understand that a compound in a relatively pure state can be also used as a “reference standard.” A reference standard is similar to a reference marker, which is used for qualitative analysis only, but is used to quantify the amount of the compound of the reference standard in an unknown mixture, as well. A reference standard is an “external standard,” when a solution of a known concentration of the reference standard and an unknown mixture are analyzed using the same technique. (Strobel p. 924, Snyder p. 549, Snyder, L. R.; Kirkland, J. J. Introduction to Modern Liquid Chromatography, 2nd ed. (John Wiley & Sons: New York 1979)). The amount of the compound in the mixture can be determined by comparing the magnitude of the detector response. See also U.S. Pat. No. 6,333,198, incorporated herein by reference.

The present invention provides a method for preparing crystalline forms of tegaserod maleate having an amount of the TGS-dimer of less than about 0.02% by area percent HPLC. This method includes starting with a tegaserod maleate sample comprising a sufficiently low level of TGS-dimer. Preferably, the amount of TGS-dimer in the tegaserod maleate sample is about 0.02% or more by area percent HPLC.

Preferably, the tegaserod maleate is in crystalline forms.

The crystalline forms prepared according to the method provided herein may be the crystalline forms described in co-pending US 2005/0165085A1 published on Jul. 28, 2005, i.e., crystalline forms A, B, B1, B2, B3, C, D, E, F, H and J.

The method provided in the present invention comprises;
a) obtaining one or more samples of one or more TGS maleate batches;
b) measuring the level of TGS-dimer in each of the samples of (a);
c) selecting the TGS maleate batch that comprises a level of the tegaserod dimer of less than about 0.02% by area percent HPLC based on the measurement or measurements conducted in (b); and
d) using the batch selected in (c) to prepare said crystalline forms of TGS maleate.

This invention also provides a method of preparing a composition comprising crystalline form of TGS maleate, having TGS-dimer in an amount of less than about 0.02% by area percent HPLC, which method comprises;
a) purifying a composition comprising TGS maleate and TGS-dimer until a composition comprising less than about 0.02% TGS dimer by area percent HPLC is obtained; and
b) using the composition resulting from (a) to prepare a composition comprising a crystalline form of TGS maleate.

Methods of purification of the composition of TGS maleate and TGS-dimer that can be used in this invention include the method described above.

The present invention further provides a method of preparing a composition comprising a crystalline form of TGS maleate, that comprises TGS-dimer in an amount of less than about 0.02% by area percent HPLC. This method comprises;

a) obtaining one or more samples of one or more TGS maleate batches;

b) measuring the level of TGS-dimer in each of the samples of (a);

c) if the quantity of the TGS-dimer measured in b) is about 0.02% area by HPLC or more, purifying the sample until the quantity of the TGS-dimer is less than about 0.02% by weight, and synthesizing a crystalline form of TGS maleate from the sample so purified; or

d) if the quantity of TGS-dimer measured in b) is less than about 0.02% by weight, synthesizing a crystalline form of TGS maleate from the TGS maleate of step b).

The purifying in step c) may be performed according to the purification processes of TGS maleate described above.

Having described the invention with reference to certain preferred embodiments, other embodiments will become apparent to one skilled in the art from consideration of the specification. The invention is further defined by reference to the following examples describing in detail the preparation of the composition and methods of use of the invention. It will be apparent to those skilled in the art that many modifications, both to materials and methods, may be practiced without departing from the scope of the invention.

### EXAMPLES

### Example 1

### Determination of Impurities in a Composition of Tegaserod Maleate

Determining the level of impurities in tegaserod maleate using HPLC. The HPLC detection conditions are as described in Table 3.

TABLE 3
HPLC method for detecting the level of the impurities

| Column & Packing: | Zorbax, Bonus RP, 5μ, 250 × 4.6 mm | | |
|---|---|---|---|
| Buffer: | (NH₄)H₂PO₄ (0.04M) pH-5.0 | | |
| | (adjusted with H₃PO₄ 5N) | | |
| Eluent A: | 75% (NH₄)H₂PO₄ (0.04M) pH-5.0 | | |
| | (adjusted with H₃PO₄, 5N) | | |
| | 25% ACN (gradient grade) | | |
| Eluent B: | Acetonitrile (gradient grade) | | |
| Gradient: | Time | % Eluent A | % Eluent B |
| | 0 | 100 | 0 |
| | 3 | 100 | 0 |
| | 63 | 53 | 47 |

| | |
|---|---|
| Equilibrium time: | 10 min |
| Sample volume: | 50 μL |
| Flow Rate: | 1.0 mL/min. |
| Detector: | 225 nm |
| Column temperature | 25° C. |
| Diluent: | Buffer-60% + ACN-40% |
| Detection limit | 0.02% by area |

Preparation of Standard Solution

Preparing a standard solution of TGS maleate having the final concentration of 0.0004 mg/mL. The Standard solutions have to be prepared and injected immediately.

Preparation of Sample Solution

Preparing a solution of TGS maleate having a final concentration of about 0.4 mg/mL in a diluent. The standard solutions have to be prepared and injected immediately.

Procedure for detecting the impurities

Inject standard and sample solutions into the chromatograph and continue the chromatogram up to 63 min. The areas of the impurities peaks in each solution is determined using a suitable integrator. Peaks which appear in a diluent place and peak with RRT 1.06, if detected, must be disregarded (it is identified as TGS′ tautomer).
Calculation

$$\% imp i = \frac{Area of imp i}{Average response factor of imp i \times conc . sample} \times potency of TGS standard$$

In the above calculation imp i identifies an impurity measured/detected.

### Example 2

### Purification of Tegaserod Maleate in Ethyl Acetate/Water (70° C.)

To a mixture of 10 g TGS-maleate in 280 mL ethyl acetate/water (1:1) was added 25.6 g of NaOH (47%) and stirred at room temperature for 24 hours. The resulting precipitate was filtrated and washed with 210 mL water (3×70 mL). To a mixture of the resulting solid in 60 mL EtOAc at 70° C., was added a solution of 3.33 g maleic acid in 30 mL ethyl acetate/water (95:5) during ½ hour, stirred at the same temperature for an additional 2 hrs, and at room temperature for overnight. The solid was filtered off and washed with ethyl acetate/water 95:5 (31.5 mL in three portions). After drying on vacuum oven at 45° C. for 15 hrs, 9.18 g of TGS maleate were obtained as an off white powder (chemical yield: 93.36%, purity: 99.96%).

### Example 3

### Purification of Tegaserod Maleate in Ethyl Acetate/Water (Room Temperature)

To a mixture of 4 g TGS-maleate in 112 mL ethyl acetate/water (1:1) was added 10.3 g of NaOH (47%) and stirred at room temperature for 24 hours. The resulting precipitate was filtrated and washed with 84 mL water (3×28 mL). To a mixture of the resulting solid in 24 mL ethyl acetate was added a solution of 1.33 g maleic acid in 13 mL ethyl acetate/water (95:5) during ½ hour, and stirred overnight. The solid was filtered off and washed with ethyl acetate/water 95:5 (30 mL in three portions). After drying on vacuum oven at 45° C. for 15 hrs, 3.33 g of TGS maleate were obtained as an off white powder (chemical yield: 83.25%, purity: 99.97%).

### Example 4

### Purification of Tegaserod Maleate in Isobutyl Acetate/Water (70° C.)

To a mixture of 4 g TGS-maleate in 112 mL isobutyl acetate/water (1:1) was added 10.3 g of NaOH (47%) and stirred at room temperature for 24 hours. The resulting precipitate was filtrated and washed with 84 mL water (3×28 mL). To a mixture of the resulting solid in 60 mL ethyl acetate at 70° C., was added a solution of 1.33 g maleic acid in 13 mL ethyl acetate/water (95:5) during ½ hour, stirred at the same temperature for an additional 2 hrs, and overnight at room temperature. The solid was filtered off and washed with ethyl acetate/water 95:5 (31.5 mL in three portions). After drying on vacuum oven at 45° C. for 15 hs, 2.58 g of TGS maleate were obtained as an off white powder (chemical yield: 64.50 %, purity: 99.98 %).

### Example 5

### Purification of Tegaserod Maleate with Acetic Acid

To a mixture of 1 g TGS maleate in 40 mL ethanol/water (3:1) was added 2.6 g of NaOH (47%) followed by 3.45 mL acetic acid and stirred at room temperature for 24 hours. The resulting precipitate was filtrated and washed with 30 mL water. To a mixture of the resulting solid in 8 mL ethyl acetate was added a solution of 0.33 g maleic acid in 3 mL ethyl acetate/water (95:5) during 20 minutes, and stirred overnight. The solid was filtered off and washed with ethyl acetate/water 95:5 (30 mL in three portions). After drying on vacuum oven at 45° C. for 15 hs, 0.73 g of TGS maleate were obtained as an off white powder (chemical yield: 73.00%, purity: 99.95%).

Synthetic Processes

### Example 6

### Preparation of TGS-Dimer

A three neck flask equipped with mechanical stirrer and thermometer, was charged with a mixture of 10 g of diaminoguanidine HCl in 250 mL of water, 30.70 g of MICHO followed by HCl (37%) until pH 2-3. The mixture was stirred at 70° C. for half hour and at room temperature for overnight. The resulting solid was filtrated and washed with 140 mL water (2×70 mL) and triturated in 250 mL ethyl acetate for 2 hours. After filtration and washing with ethyl acetate (2×125 mL) the wet material was dried on vacuum oven (45° C., overnight) giving TGS-dimer (32 g, 91% yield, 98% purity by HPLC).

1. A method of purifying Tegaserod maleate comprising;
a) combining tegaserod maleate, a first organic solvent and an inorganic base to obtain a reaction mixture;
b) maintaining the reaction mixture at a temperature of from about room temperature to about the boiling temperature of the reaction mixture to obtain a precipitate;
c) combining the precipitate with a second organic solvent and a maleic acid with or without water at a temperature of from about room temperature to about the boiling temperature of the mixture; and
d) recovering the purified Tegaserod maleate.

2. A method of purifying Tegaserod maleate comprising;
a) combining tegaserod maleate and a first organic solvent to obtain a reaction mixture;
b) combining the reaction mixture with an inorganic base and an organic carboxylic acid;
c) maintaining the reaction mixture obtained in step b) at a temperature of from about room temperature to about the boiling temperature of the reaction mixture to obtain a precipitate;
d) combining the precipitate with a second organic solvent and a solution of maleic acid, the second organic solvent, and water at a temperature of from about room temperature to about the boiling temperature of the mixture; and
e) recovering the purified Tegaserod maleate.

3. The method of any one of claims 1 and 2, wherein the reaction mixture is maintained at about room temperature.

4. The method of claim 1, wherein the first organic solvent is a C₃-C₇ ester.

5. The method of claim 4, wherein the first organic solvent is selected from the group consisting of isobutyl acetate and ethyl acetate.

6. The method of any one of claims 1 and 2, wherein the inorganic base is in an aqueous solution.

7. The method of claim 2, wherein the first organic solvent is selected from the group consisting of a C₁-C₄ alcohol, a C₆-C]₂ aromatic solvent, a C₁-C₇ alkane or a C₂-C₈ ether.

8. The method of claim 7, wherein the first organic solvent is ethanol.

9. The method of any one of claims 1 and 2, wherein the mixture of the first organic solvent and water is 1:1 volumes.

10. The method of any one of claims 1 and 2, wherein the amount of inorganic base added is 10 mol equivalents

11. The method of any one of claims 1 and 2, wherein the second organic solvent is selected from the group consisting of: a C₁-C₈ alcohol, C₁-C₄ ketones, C₁-C₇ ethers, C₃-C₇ esters, acetonitrile, dioxane and tetrahydrofuran.

12. The method according to claim 11, wherein the second organic solvent is selected from the group consisting of methanol, ethanol, isopropanol, acetonitrile, butanol, acetone, dioxane, methylethyl ketone, tetrahydrofurane, ethyl lactate, and ethyl acetate.

13. The method according to claim 11, wherein the second organic solvent is ethyl acetate.

14. The method of claim 1, wherein the reaction mixture is maintained in step b) for a period of more than about 2 hours.

15. The method of claim 2, wherein the reaction mixture is maintained in step c) for a period of more than about 5 hours.

16. The method of claim 2, wherein the organic carboxylic acid of step b) has a pKa higher than about 2.

17. The method of claim 16, wherein said organic carboxylic acid is acetic acid.

18. The method of any one of claims 1 and 2, wherein the precipitate is combined with the second organic solvent at a temperature of about room temperature to about 70° C.

19. The method of any one of claims 1 and 2, wherein combining the precipitate with a second organic solvent and a maleic acid is performed in the presence of water.

20. The method of any one of claims 1 and 2, wherein recovering of the purified tegaserod maleate comprises filtration.

21. The method of any one of claims 1 and 2, further comprising drying the purified tegaserod maleate.

22. The method of any one of claims 1 and 2, wherein the purified tegaserod maleate contains an amount of less than about 0.02% area by HPLC of the dimer impurity and of the impurities at RRT 2.01 and RRT 0.89.

23. An isolated compound of the formula I; 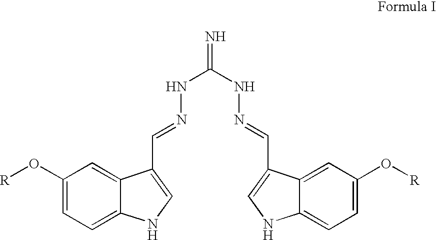
wherein R is selected from the group consisting of: saturated and unsaturated branched and linear C₁-C₄ alkanes, C₁-C₄ ethers, C₁-C₃ alcohols, C₆-C₁₀ aromatic hydrocarbons and amides.

24. The isolated compound of claim 23, wherein R is selected from the group consisting of: CH₂OCH₃, CH₂CH═C(CH₃)₂, CO—N(CH₃)₂, CH₃, C₂H₅, C₃H₇, benzoyloxy, CO-tert-C₄H₉, CO—N(C₂H₅)₂, CH₂—CO—N(CH₃)₂, CH₂—CH₂—N(CH₃)₂, (CH₂)₂—O—CH₃, (CH₂)₂—OH, isobutoxy, 2, 3-di(OH)-propoxy and acetoxy.

25. The isolated compound of claim 24, wherein R is methyl.

26. The isolated compound of claim 25, characterized by data selected from: ¹H NMR (500 MHz, DMSO d₆) δ(ppm): 11.73 (s, 2H), 8.53 (s, 2H), 7.89 (s, 4H), 7.75 (s, 2H), 7.37 (d, J=8.7 Hz, 2H), 6.87 (d, J=7.9 Hz, 2H), 3.87 (s, 6H); by ¹³C{¹H}NMR analysis (125 MHz) δ (ppm): 154.83, 152.03, 146.32, 132.18, 124.76, 112.73, 112.65, 110.48, 104.41, 55.56; by FAB mass spectrometry analysis showing the following data: m/z=404.1 ([M−H]⁺, 100%) and by IR(KBr) ν cm⁻¹: 3385 (N—H), 2944 (C—H), 1641, 1637, 1613 (C═N), 1528, 1485 (Cₐᵣ-Cₐᵣ).

27. A method of using the isolated compound of claim 25 as a reference marker.

28. The method of claim 27, wherein the reference marker is an HPLC marker or an indicator for purity in a process.

29. A method of using the isolated compound of claim 25 as a reference standard.

30. A method for preparing the isolated compound of claim 23, comprising the steps of;
a) mixing dianinoguanidine-HCl with water;
b) adding a compound of formula II; 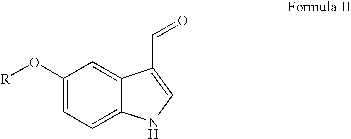
and an acid until a reaction mixture having a pH of about 2 to about 3 is obtained, wherein R is saturated and unsaturated, branched and linear C₁-C₄ alkanes, C₁-C₄ ethers, C₁-C₃ alcohols, C₆-C₁₀ aromatic hydrocarbons and amides;
c) maintaining the reaction mixture at a temperature from about room temperature to about boiling temperature of reaction mixture to obtain a precipitate; and
d) recovering a tegaserod derivative.

31. The method of claim 30, wherein R is selected from the group consisting of:
CH₂OCH₃, CH₂CH═C(CH₃)₂, CO—N(CH₃)₂, CH₃, C₂H₅, C₃H₇, benzoyloxy, CO-tert-C₄H₉, CO—N(C₂H₅)₂, CH₂—CO—N(CH₃)₂, CH₂—CH₂—N(CH₃)₂, (CH₂)₂—O—CH₃, (CH₂)₂—OH, isobutoxy, 2,3-di(OH)-propoxy and acetoxy.

32. The method of claim 30, wherein R is methyl.

33. The method of to claim 30, wherein the acid is an inorganic acid selected from the group consisting of HCl, HBr, H₂SO₄, and H₃PO₄, or a carboxylic acid

34. The method of to claim 33, wherein the acid is HCl.

35. The method of claim 30, wherein the reaction mixture is maintained at about 70° C.

36. A method for preparing crystalline forms tegaserod maleate having an amount of the impurity tegaserod dimer (TGS-dimer) of less than about 0.02% by area percent HPLC comprising;
a) obtaining one or more samples of one or more tegaserod maleate batches;
b) measuring the level of tegaserod dimer in each of the samples of (a);
c) selecting the tegaserod maleate batch that comprises a level of the tegaserod dimer of less than about 0.02% by area percent HPLC based on the measurement or measurements conducted in (b); and
d) using the batch selected in (c) to prepare said tegaserod maleate in crystalline form.

37. A method of preparing a composition comprising crystalline form of TGS maleate, having TGS-dimer in an amount of less than about 0.02% by area percent HPLC, which method comprises;
a) purifying a composition comprising TGS maleate and TGS-dimer until a composition comprising less than about 0.02% TGS dimer by area percent HPLC is obtained; and
b) using the composition resulting from (a) to prepare a composition comprising a crystalline form of TGS maleate.

38. The method according to claim 37, wherein the purifying a composition comprising tegaserod maleate and a tegaserod dimer is performed according to claims 1 or 2.

39. A method of preparing a composition comprising crystalline form of TGS maleate, having TGS-dimer in an amount of less than about 0.02% by area percent HPLC, which method comprises;
a) obtaining one or more samples of one or more TGS maleate batches;
b) measuring the level of TGS-dimer in each of the samples of (a);
c) if the quantity of the TGS-dimer measured in b) is about 0.02% area by HPLC or more, purifying the sample until the quantity of the TGS-dimer is less than about 0.02% by weight, and synthesizing a crystalline form of TGS maleate from the sample so purified; or
d) if the quantity of TGS-dimer measured in b) is less than about 0.02% by weight, synthesizing a crystalline form of TGS maleate from the TGS maleate of step b).

40. The method according to claim 39, wherein the purifying a composition comprising tegaserod maleate and a tegaserod dimer is performed according to claims 1 or 2.
